(19) United States
(12) Patent Application Publication
(10) Pub. No.: US 2026/0000830 A1
(43) Pub. Date: Jan. 1, 2026

(54) INFUSION PUMP, INFUSION SYSTEM AND METHOD

(71) Applicant: B. Braun Melsungen AG, Melsungen (DE)

(72) Inventor: Jan Schwarz, Melsungen (DE)

(21) Appl. No.: 19/248,367

(22) Filed: Jun. 24, 2025

(30) Foreign Application Priority Data
Jul. 1, 2024 (EP) 24185764.8

(51) Int. Cl. A61M 5/172; A61M 5/142; G06F 21/44; G16H 20/17; G16H 40/63

(52) U.S. Cl. CPC A61M 5/172; A61M 5/14228; G06F 21/44; G16H 20/17; G16H 40/63; A61M 2205/3306; A61M 2205/3317; A61M 2205/3584; A61M 2205/6063

(57) ABSTRACT

An infusion pump is configured for use with at least one infusion item. The infusion pump includes at least one authentication device for authenticating the at least one infusion item for use with the infusion pump.

### CROSS-REFERENCE TO RELATED APPLICATION

This application claims priority under 35 U.S.C. § 119 to European Application No. 24185764.8, filed on Jul. 1, 2024, the content of which is incorporated by reference herein in its entirety.

### FIELD

The present disclosure relates to an infusion pump, an infusion system and a method for operating an infusion pump.

### BACKGROUND

Single-use/disposable infusion devices are essential components in healthcare that are used in conjunction with infusion pumps to ensure the administration of fluids, medications and nutrients to patients. These devices, which typically include sterile infusion sets, tubing and bags, as well as syringes, are specifically designed to be used only once to minimize the risk of cross-contamination and infection.

Infusion pumps, which may be built as peristaltic or syringe pumps, are designed to deliver precise and controlled amounts of fluids into a patient's bloodstream. The integration of disposable infusion products, in particular tubes, bags and syringes, into these pumps not only ensures a high degree of accuracy and safety in dosing, but also improved patient care and safety. The use of these disposable products thus plays a central role in modern medical practice, enabling sterile and efficient drug administration.

It is known from WO 2015/150280 A1 how to monitor the configuration and condition of an infusion system. For this purpose, a signal generator is used to actively introduce a signal into the liquid, which is transmitted through the liquid and detected at another location, at a distance from the signal generator, by means of a sensor.

### SUMMARY

The present disclosure is based on the object of providing an infusion pump that avoids the disadvantages of the prior art. In particular, only the use of infusion articles certified for this purpose with the infusion pump should be made possible. Furthermore, an improved infusion system and an improved method for operating an infusion pump should be provided.

With regard to the infusion pump, this object is solved by an infusion pump designed for use with at least one infusion article or disposable infusion article. The infusion pump has at least one authentication device for authenticating the at least one infusion article.

The present disclosure is based on the consideration that, for reliable patient care and to avoid contamination, it is important that the infusion article is certified as approved for the infusion pump, since only in this way can patient harm be reliably averted. The infusion article must be qualified for the infusion pump and thus be an authenticated infusion article.

It has now been recognized that patient harm caused by unsuitable infusion items can be reliably prevented by first authenticating the infusion item with the infusion pump before putting it into operation.

The at least one authentication device advantageously has a sensor. The sensor can be used to detect an authentication feature arranged on the infusion item.

The sensor is preferably an electromagnetic sensor. Advantageously, the sensor is designed as an optical sensor, in particular as a camera. Alternatively, the electromagnetic sensor can be designed as an RFID reader or NFC sensor.

In an alternative preferred embodiment, the at least one authentication device is designed as a mechanical locking device. Such a design offers less copy protection than the use of electromagnetic sensors and requires mechanical methods to be able to recognize an infusion article. The mechanical coding can be built in the form of raised features or holes/perforated strips that are activated by pressed springs/tasters, thus authenticating the infusion article.

The authentication of the infusion article preferably includes the detection of at least one authentication feature arranged on the infusion article. The detection can be carried out by means of a sensor or also mechanically, for example by means of a key-lock mechanism. In the case of an optical sensor, the detection is carried out, for example, by detecting a QR code, in particular by scanning the QR code. The authentication of the infusion article preferably also includes a comparison of the authentication feature or the information contained therein with a reference, in particular a database with information on infusion articles that are certified for use with the infusion pump.

In a first preferred embodiment, the infusion pump is configured as a syringe pump. In a second preferred embodiment, the infusion pump is configured as a peristaltic pump.

With regard to the infusion system, the above-mentioned object is solved by an infusion system comprising an infusion pump as described above and an infusion article, wherein the infusion article has at least one authentication feature that can be detected and authenticated by the at least one authentication device.

As soon as an infusion article is inserted into the infusion pump, the sensor recognizes the authentication feature applied to the infusion article and reads out the associated data, that is, the sensor detects the authentication feature. The data read in this way is checked against the certificate/validity table created during the manufacturing of the infusion article. If the read data matches the data stored in the certificate/validity table, the infusion article is considered authenticated and can be used in the infusion pump.

Otherwise, the infusion pump will not allow the user to use this infusion article, thus ensuring that only a certified infusion article, i.e. one that has been approved by the manufacturer, is used. This is particularly necessary to achieve the stated flow rate and its tolerance in the infusion pump with the infusion article and thus ensure the quality of the product. The data synchronization can take place in the infusion pump, but it preferably takes place on a server that stores this data so that the validity table remains in the control of the manufacturer.

The infusion article is, for example, an infusion tube or a syringe.

The authentication feature includes information that allows a unique certification or validity of the infusion article in the infusion pump. These are in particular serial number/consecutive number, lot number, production date, production location, buyer and/or manufacturer. The authentication feature is preferably designed as a QR code, which is attached to the infusion article, for example, in particular by screen printing or laser, printed or glued on.

Alternatively, the authentication feature can be designed as an RFID tag, which is arranged on the infusion article.

The infusion pump is advantageously designed to authenticate the detected authentication feature by matching it with a database.

The database preferably has a certificate table or validity table or is designed as such a table.

The database can be stored in the infusion pump itself or on a server.

In a first preferred embodiment, the database is stored in the infusion pump. This allows authentication to be carried out directly by the infusion pump. The certificates/validity tables are stored in the infusion pump before the buyer uses the infusion article. When an infusion article is inserted into the infusion pump and the authentication data is read from the infusion disposable article, the read data is authenticated against the data stored in the certificate/validity table. If its validity is confirmed, the infusion article can be used in the infusion pump, otherwise not.

In a second preferred embodiment, the infusion system includes a server, the database being stored on the server, and the infusion pump being designed to send a request to the server for a recorded authentication feature, and the server being designed to send a response to the infusion pump which contains whether the information contained in the authentication feature is contained in the database.

Centralized authentication using a server of the respective infusion device by the infusion pump is preferable to local authentication, in which the database is stored in the infusion pump, because it is less expensive, but requires a strong network and thus a communication method within the infusion pump. The infusion pump advantageously has a communication module for this purpose, which is designed to establish a bidirectional communication channel to the server for exchanging information. The communication module is preferably based on Wi-Fi/WLAN, mobile radio or satellite communication. The database is stored on this server.

After reading the authentication data from the infusion article, the infusion pump establishes a communication channel to the server and authenticates/verifies the read data for validity. If the data is valid, the infusion article can be used without restriction. Otherwise, the infusion pump rejects the inserted infusion article and denies its use.

The infusion pump is advantageously designed to reject the use of the infusion article if the authentication feature is not present in the database, and otherwise to allow it. The infusion article is preferably provided with a QR code, in which a lot number and/or a serial number and the manufacturer are preferably stored. This information is advantageously read by the infusion pump via a sensor, in particular a camera, and then checked for validity in the form of a request to a server. The infusion pump compares whether the infusion article has already been used before (including a certain grace period) and rejects use if the infusion article has already been used.

The database preferably contains encrypted or encoded authentication data. In this case, the infusion pump's authentication device is designed to recognize the correctness of the certificates/validity tables that have been uploaded, for example by means of private/public keys. Invalid certificates are rejected/not processed by the infusion pump.

With regard to the procedure, the above-mentioned object is solved according to the present disclosure by placing an infusion article in the infusion pump and/or connecting it to it, and the infusion pump checks whether the infusion article is approved for use with the infusion article by comparing it with a certificate database.

The advantages of the present disclosure lie in particular in the fact that an optimized patient care can be reliably provided by the possibility of authenticating an infusion article prior to the commissioning of the infusion pump, and patient damage, which may occur due to the use of non-certified articles, can be prevented in advance.

### BRIEF DESCRIPTION OF THE DRAWINGS

An embodiment of the present disclosure is explained in more detail in a drawing. In this drawing, in a highly schematized representation, the following is shown:

FIG. 1 a peristaltic pump with a patient supply tube inserted;

FIG. 2 a syringe pump with an infusion tube inserted;

FIG. 3 a syringe with a QR code applied;

FIG. 4 an infusion tube with a QR code applied;

FIG. 5 an infusion pump with an infusion article inserted, and

FIG. 6 an illustration of the data flow for authentication with a server.

The same parts are provided with the same reference signs in all figures.

### DETAILED DESCRIPTION

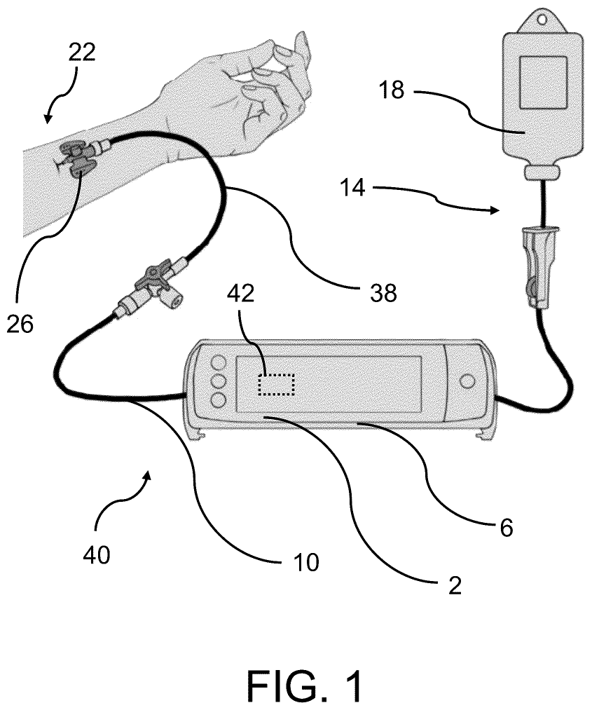

FIG. 1 shows an infusion pump 2 which is designed as a peristaltic pump 6 or hose pump, i.e. as a positive displacement pump in which the medium to be delivered is pressed through a hose by external mechanical deformation of the hose. An infusion tube 10 is inserted into the peristaltic pump 6, which is connected at a first end 14 to an infusion bag 18 with infusion solution and is connected at a second end 22 to a cannula 26. The peristaltic pump 6 conveys infusion solution through the infusion tube 10, so that infusion solution is supplied to the patient at a predetermined dosage rate.

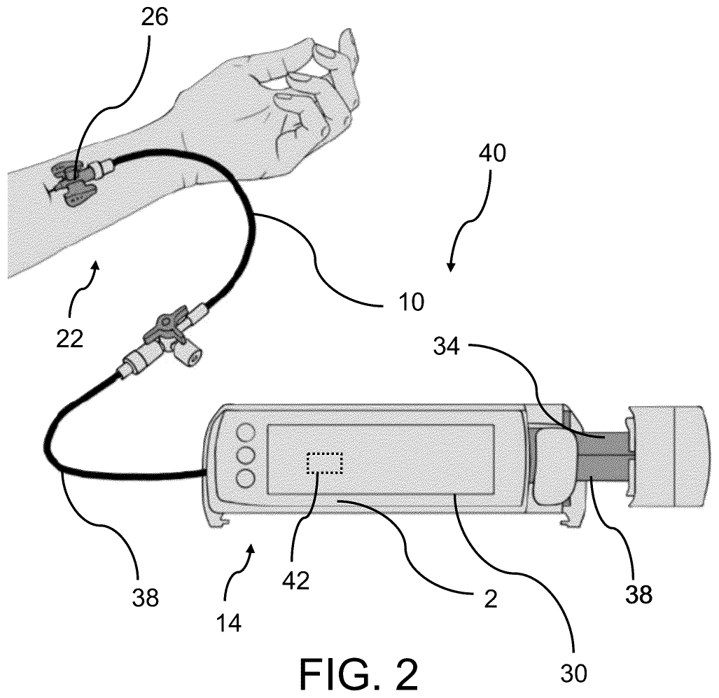

FIG. 2 shows an infusion pump 2 which is designed as a syringe pump 30. A syringe 34 is inserted into the syringe pump 30, which is connected at a first end 14 (in the syringe pump 30) to an infusion tube 10 and at a second end 22 to a cannula 26.

The infusion tube 10 and the syringe 34 are infusion devices 38 used together with the infusion pump 2. In FIG. 1 and FIG. 2, the infusion pump 2 and infusion article 38 each constitute an infusion system 40. Reliable patient care is only possible if only an infusion article 38 that is certified or validated for use with the respective infusion pump 2 is used. Only certified infusion devices 38 should be used with the infusion pump 2; it should not be possible to operate the infusion pump 2 with a non-certified infusion device 38.

For this purpose, the infusion pump 2 is provided with an authentication device 42 (shown dashed, inside the infusion pump) that authenticates the infusion article 38. The authentication device 42 comprises a sensor 46, which in this case is designed as a camera.

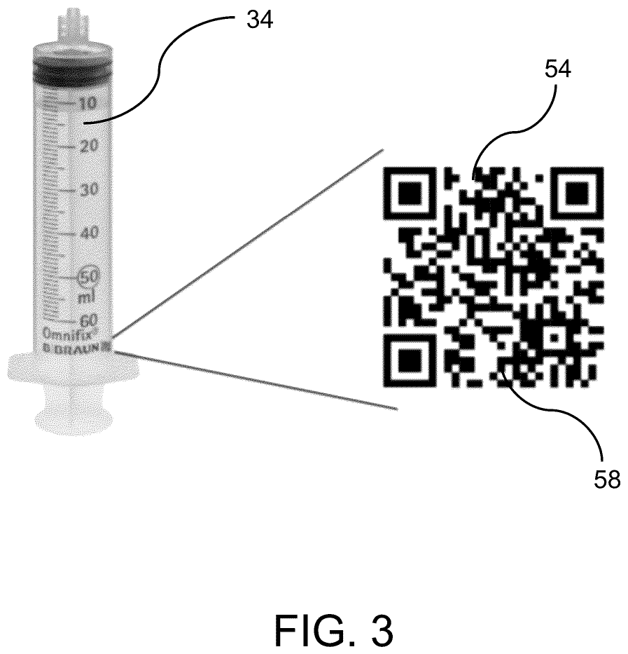

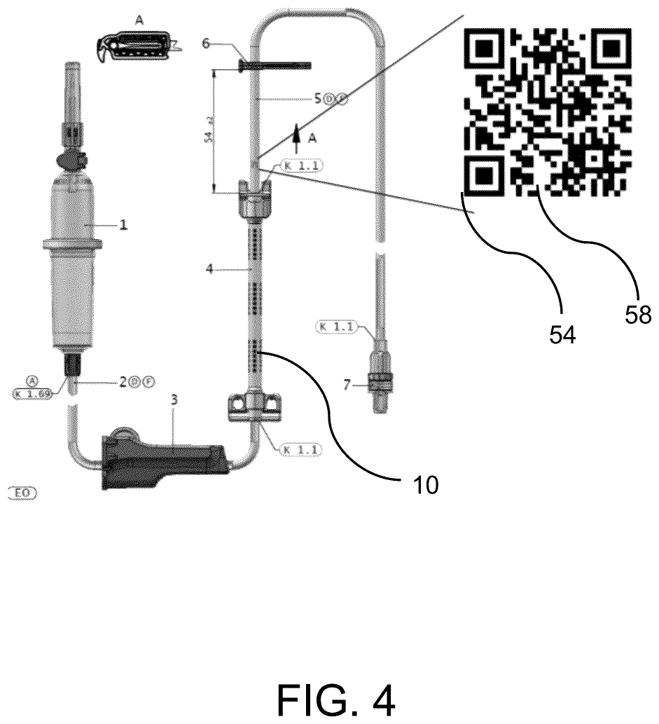

The infusion article 38 comprises at least one authentication feature 54 for this purpose (see FIG. 3 and FIG. 4). The authentication feature 54 is authenticated by the authentication device 42, which is described in more detail below.

FIG. 3 shows an example of a syringe 34 for infusion solution. The syringe 34 has an authentication feature 54, which in this case is designed as a QR code 58 and is shown enlarged on the right in FIG. 3. In the syringe pump 30 according to FIG. 2, a camera (not shown) is arranged in such a way that when the syringe 34 is inserted for normal use, the camera is located opposite the QR code 58 so that the QR code 58 is captured by the camera.

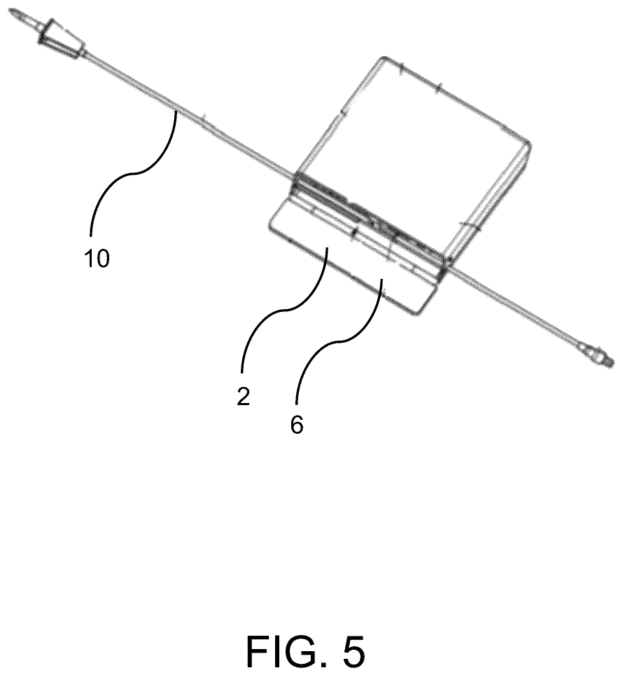

FIG. 4, for example, shows a patient supply tube 10 that has an authentication feature 54 in the form of a QR code 58, which is shown enlarged on the right in FIG. 4. FIG. 5 shows a peristaltic pump 6 with an infusion tube 10 inserted.

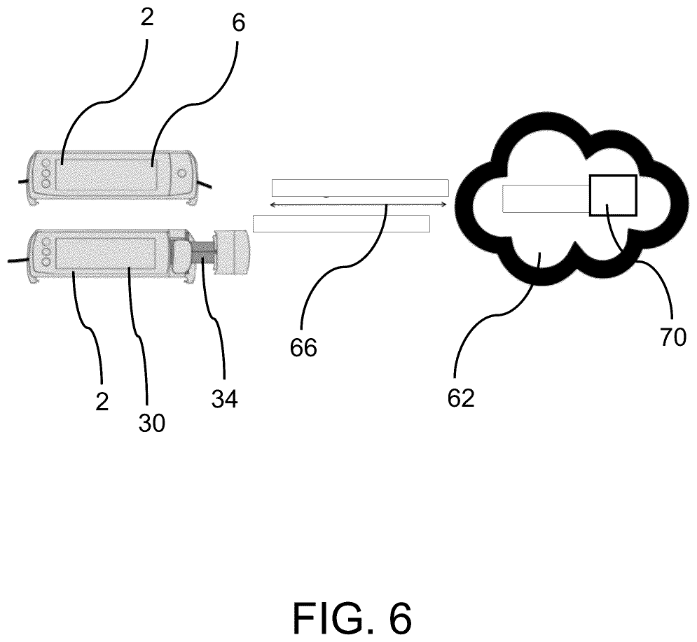

FIG. 6 schematically shows a peristaltic pump 6 and a syringe pump 30 and a server 62. A bidirectional information exchange between the respective infusion pump 2 and the server 62 is represented by a double arrow 66. For this purpose, the infusion pump 2 has a communication module for communicating with the server 62.

After the camera of the authentication device 42 of infusion pump 2 has captured the authentication feature 54 on the infusion article 38, the authentication device 42 extracts the information contained therein and sends it, preferably encrypted, to the server 62. The server 62 compares this information with the information stored in a database 70 stored or installed on the server 62. If the information transmitted by the communication module of infusion pump 2 is stored in the database 70, the server 62 signals this to the communication module of infusion pump 2 and infusion pump 2 can be operated with this infusion article 38. In this case, the infusion pump 2 allows the use of the infusion article 38. The authentication of the infusion article 38 was positive in this case.

If the above information is missing from the database and/or is incomplete, the server signals this to the communication model of infusion pump 2. In this case, the authentication of infusion article 38 was unsuccessful. The infusion pump 2 then blocks operation with this infusion article 38. This can be done, for example, by the infusion pump 2 not pumping and requiring a new insertion of the infusion article, possibly with a prompt to the user via a display. Preferably, the user is shown a display that indicates the rejection of the infusion article 38 with a reason. In addition, the information about the rejection is logged in the infusion pump 2, and, if necessary, a fine logging is also carried out on the server 62. At the same time, an evaluation of the infusion articles 38 that have already been used can be carried out via the server 62, and, if necessary, prohibit the reuse of the same infusion article 38 and/or indicate that an infusion article 38 should be used multiple times. Furthermore, it is possible that not only a display on the infusion pump 2 is shown regarding the reason for the unavailability of the infusion article, but also an error message is displayed on the server 62 (including logging). Furthermore, it is conceivable to send a warning to the manufacturer that an infusion article 38 is/was used on one of his devices that is not authenticated.

### LIST OF REFERENCE SIGNS

2 infusion pump
6 peristaltic pump
10 infusion tube
14 first end
18 infusion bag
22 second end
26 cannula
30 syringe pump
34 syringe
38 infusion item
40 infusion system
42 authentication device
46 sensor
54 authentication feature
58 QR code
62 server
66 double arrow
70 database

1. An infusion pump designed for use with at least one infusion article, the infusion pump comprising at least one authentication device for authenticating the at least one infusion article.

2. The infusion pump according to claim 1, wherein the at least one authentication device comprises a sensor.

3. The infusion pump according to claim 2, wherein the sensor is an electromagnetic sensor.

4. The infusion pump according to claim 2, wherein the sensor is designed as an optical sensor.

5. The infusion pump according to claim 4, wherein the optical sensor is a camera.

6. The infusion pump according to claim 1, wherein the at least one authentication device is a mechanical locking device.

7. The infusion pump according to claim 1, wherein the infusion pump is a syringe pump.

8. The infusion pump according to claim 1, wherein the infusion pump is a peristaltic pump.

9. An infusion system comprising:
the infusion pump according to claim 1; and
an infusion article comprising at least one authentication feature configured to be detected and authenticated by the at least one authentication device of the infusion pump.

10. The infusion system according to claim 9, wherein the infusion pump is configured to authenticate the at least one authentication feature by matching the at least one authentication feature with a database.

11. The infusion system according to claim 10, wherein the database comprises a certificate table.

12. The infusion system according to claim 11, wherein the database is stored in the infusion pump.

13. The infusion system according to claim 10, further comprising a server, wherein:
the database is stored on the server, and
the infusion pump is configured to send a request to the server with regard to a recorded authentication feature.

14. The infusion system according to claim 13, wherein the server is configured to send a response to the infusion pump, the response containing information as to whether information contained in the at least one authentication feature is contained in the database.

15. The infusion system according to claim 10, wherein the infusion pump is configured to deny use of the infusion article if the at least one authentication feature is not present in the database and otherwise to allow use.

16. The infusion system according to claim 10, wherein the database contains encrypted authentication data.

17. A method for operating an infusion pump, the method comprising the steps of:
inserting an infusion article into and/or connected to the infusion pump; and
using the infusion pump to check whether the infusion article is approved for operation with the infusion pump by matching the infusion article with a certificate database.
